(19) United States
(12) Patent Application Publication
(10) Pub. No.: US 2013/0244274 A1
(43) Pub. Date: Sep. 19, 2013

(54) SAMPLE PROCESSING APPARATUS AND REAGENT INFORMATION INFORMING METHOD

(71) Applicant: SYSMEX CORPORATION, Kobe-shi (JP)

(72) Inventors: Ken NISHIKAWA, Kobe-shi (JP); Yuichi HAMADA, Kobe-shi (JP)

(73) Assignee: SYSMEX CORPORATION, Kobe-shi (JP)

(21) Appl. No.: 13/777,528

(22) Filed: Feb. 26, 2013

(30) Foreign Application Priority Data
Mar. 16, 2012 (JP) 2012-060303

(51) Int. Cl. G01N 33/50

(52) U.S. Cl. CPC G01N 33/50
USPC 435/39; 422/67; 436/174; 435/287.1

(57) ABSTRACT

A sample processing apparatus comprises a sample processing unit that carries out processing of a sample using a reagent, an acquiring device that acquires reagent information regarding a reagent in a reagent container provided to the reagent container, an audio output unit that outputs audio, and a control device that manages the reagent information of the reagent in a state of being used by the sample processing unit. The audio output unit outputs audio guidance regarding the acquired reagent information when the acquiring device acquires new reagent information regarding a reagent not in a state of being used by the sample processing unit.

### RELATED APPLICATIONS

This application claims priority under 35 U.S.C. §119 to Japanese Patent Application No. 2012-060303 filed on Mar. 16, 2012, the entire content of which is hereby incorporated by reference.

### FIELD OF THE INVENTION

The present invention relates to a sample processing apparatus for processing samples such as blood and urine using a reagent, and a reagent information informing method.

### BACKGROUND OF THE INVENTION

A sample processing apparatus for processing samples such as blood and urine described in U.S. Patent Application Publication No. 2010/0248289 uses a plurality of reagents in sample processing. The reagent is contained in a reagent container, and the small reagent container is installed inside the sample processing apparatus. The large reagent container is installed outside the sample processing apparatus, and is connected to the sample processing apparatus by way of a fluid feeding tube. When replacing the large reagent container, identification information of the reagent contained in a new reagent container and reagent information such as the expiration date need to be registered in a control computer of the sample processing apparatus. The reagent information may be manually input by a user, but a method of inputting by reading a barcode attached to the reagent container with a barcode reader is proposed as a simpler and more convenient method.

In the conventional device, after the barcode is read by the barcode reader, the read information is displayed on a screen of the control computer. The user checks whether or not the barcode is accurately read, whether the type of read reagent is correct, whether the expiration date has not passed, and the like by looking at the displayed screen. If the read information is correct, the user pushes an OK button displayed on the screen. The read information is thereby registered in the control computer.

However, if the sample processing apparatus enlarges, the large reagent container may be installed at a position distant from the control computer. In this case, the user needs to move from the installed position of the reagent container to the control computer to check whether the information read from the barcode of the reagent container is correct.

### SUMMARY OF THE INVENTION

The scope of the present invention is defined solely by the appended claims, and is not affected to any degree by the statements within this summary.

A first aspect of the present invention is a sample processing apparatus, comprising a sample processing unit that carries out processing of a sample using a reagent, an acquiring device that acquires reagent information regarding a reagent in a reagent container provided to the reagent container, an audio output unit that outputs audio, and a control device that manages the reagent information of the reagent in a state of being used by the sample processing unit, wherein the audio output unit outputs audio guidance regarding the acquired reagent information when the acquiring device acquires new reagent information regarding a reagent not in a state of being used by the sample processing unit.

A second aspect of the present invention is a reagent information informing method, comprising (a) processing a sample using a reagent, (b) acquiring reagent information regarding a reagent in a reagent container provided to the reagent container, and (c) outputting audio, wherein when new reagent information regarding a reagent not in a state of being used in the sample processing step is acquired by an acquiring device in the acquiring step, audio guidance regarding the acquired reagent information is output in the step (c).

A third aspect of the present invention is a sample processing apparatus, comprising a sample processing unit that carries out processing of a sample using a reagent, an acquiring device that acquires reagent information regarding a reagent in a reagent container provided to the reagent container, an audio output unit that outputs audio; and a control device that manages the reagent information of the reagent in a state of being used by the sample processing unit, wherein the audio output unit outputs audio guidance regarding the acquired reagent information when the acquiring device acquires new reagent information regarding a reagent in a state of being used by the sample processing unit.

### BRIEF DESCRIPTION OF THE DRAWINGS

FIG. 1 is a view schematically showing a configuration of a sample processing system according to an embodiment when viewed from an upper side;

FIG. 2 is a view schematically showing the configuration of the sample processing system according to the embodiment when viewed from the front;

FIGS. 3A and 3B are views showing a state in which a reagent container is connected to a measurement unit, and a view showing a configuration of a barcode label;

FIG. 4 is a view showing a configuration of the measurement unit and an information processing unit according to the embodiment;

FIG. 5 is a view showing a menu screen of the information processing unit displayed on a display input unit according to the embodiment;

FIG. 6 is a view showing a menu screen in a state a help dialogue is opened according to the embodiment;

FIG. 7 is a view showing a menu screen in a state a reagent replacement dialogue is opened according to the embodiment;

FIGS. 8A-8D are conceptual diagrams showing a configuration of a reagent management DB according to the embodiment;

FIG. 9 is a flowchart showing processes performed by the information processing unit according to the embodiment;

FIG. 10 is a flowchart showing processes performed by the information processing unit according to the embodiment;

FIG. 11 is a view showing an audio guidance content output from a speaker according to the embodiment;

FIGS. 12A and 12B are views showing a reagent replacement dialogue when the reagent is in acceptance standby state or received state according to the embodiment;

and FIGS. 13A and 13 B are flowcharts showing a measurement interruption process and a process of closing the reagent replacement dialogue by the information processing unit according to the embodiment.

### DETAILED DESCRIPTION OF THE PREFERRED EMBODIMENTS

The preferred embodiments of the present invention will be described hereinafter with reference to the drawings.

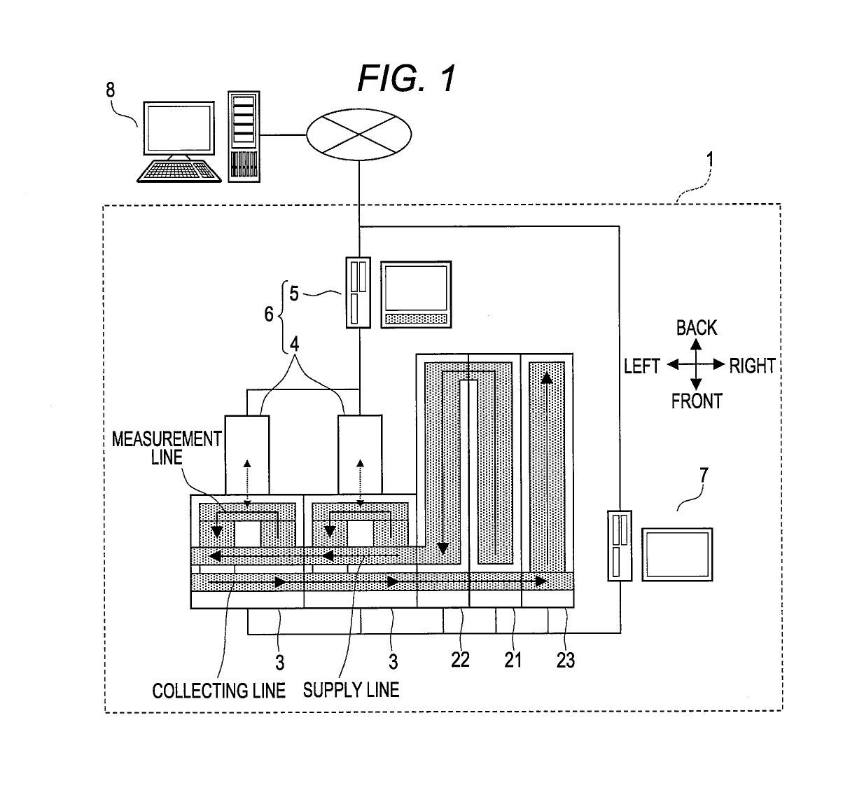

FIG. 1 is a view schematically showing a configuration of a sample processing system 1 when viewed from an upper side. The sample processing system 1 according to the present embodiment is configured by an inputting unit 21, a pre-processing unit 22, a collecting unit 23, two transportation units 3, a blood cell analyzer 6, and a transportation controller 7. The blood cell analyzer 6 is configured by two measurement units 4 and an information processing unit 5. The sample processing system 1 of the present embodiment is communicably connected with a host computer 8 through a communication network.

A sample container containing a sample is set in the inputting unit 21 while being held in a sample rack, and then transported from the inputting unit 21 to the pre-processing unit 22, and furthermore, to the measurement unit 4 through a supply line and a measurement line of the transportation unit 3. The measurement unit 4 processes the sample in the transported sample container. Thereafter, the sample container is collected by the collecting unit 23 through a collecting line of the transportation unit 3.

The measurement unit 4 takes out the sample container from the sample rack at a predetermined position (near dotted line arrow in the figure) on the measurement line to measure the sample contained in the relevant sample container in the measurement unit 4. After the measurement is completed, the sample container is again returned to the original sample rack. The measurement unit 4 includes an optical detector for carrying out optical measurement on the sample, and the optical information (lateral fluorescent signal, forward scattered light signal, lateral scattered light signal) is detected as data of the sample from each blood cell in a measurement specimen by the optical detector and output to the information processing unit 5.

The information processing unit 5 is communicably connected to two measurement units 4, and controls the operation of two measurement units 4. The information processing unit 5 is communicably connected with the host computer 8 through the communication network. The information processing unit 5 makes an inquiry of a measurement order to the host computer 8, gives a measurement instruction to the measurement unit 4 based on the inquiry result, and causes the measurement unit 4 to execute the measurement of the sample. The information processing unit 5 receives the data of the sample from the two measurement units 4 and analyzes the data, and transmits the analysis result such as the number of red blood cells and the number of white blood cells to the host computer 8.

The transportation controller 7 is communicably connected to the host computer 8 through the communication network. The transportation controller 7 controls the transporting operation of the inputting unit 21, the pre-processing unit 22, the collecting unit 23, and the two transportation units 3.

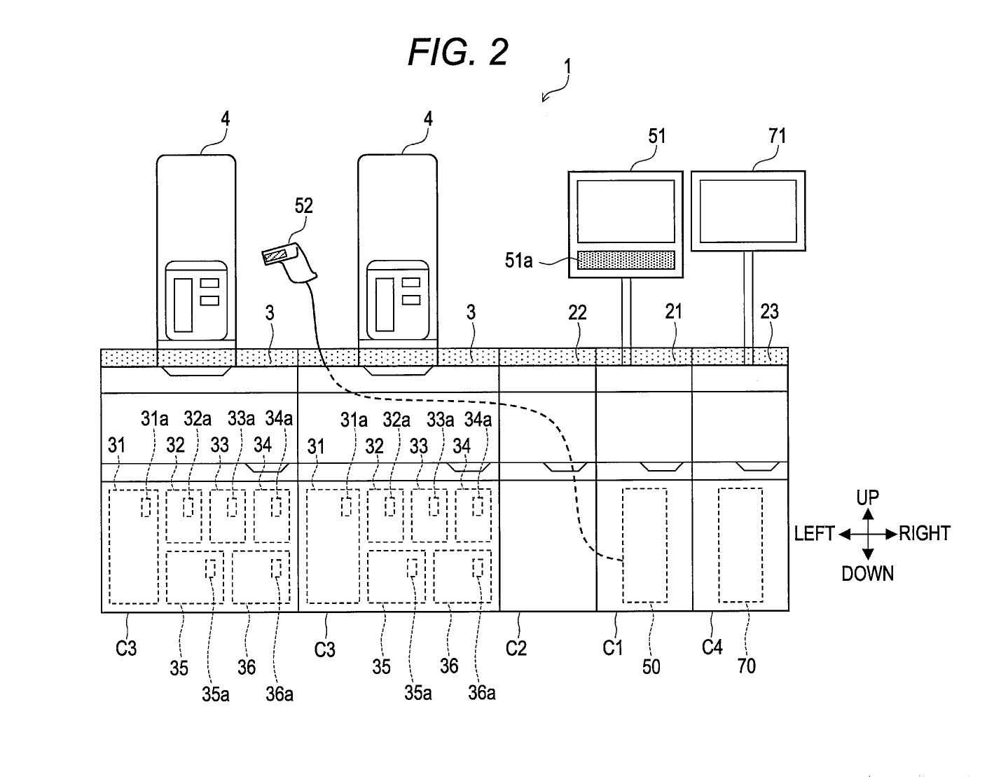

FIG. 2 is a view schematically showing the configuration of the sample processing system 1 when viewed from the front.

The information processing unit 5 includes a main body 50, a display input unit 51 including a touch panel type display, and a handy type barcode reader 52. A speaker 51a for outputting an audio signal output from the main body 50 is installed at the lower part of the display input unit 51. The transportation controller 7 includes a main body 70 and a display input unit 71 including a touch panel type display. An operator operates the display input units 51, 71 to input instruction to the main bodies 50, 70, respectively.

The inputting unit 21, the pre-processing unit 22, the transportation unit 3, and the collecting unit 23 are each installed at the upper part of carts C1 to C4. The main body 50 of the information processing unit 5 and the main body 70 of the transportation controller 7 are respectively accommodated at the lower part of the carts C1, C4. The measurement unit 4 is installed on the back side than the cart C3, and the display input unit 51 of the information processing unit 5 and the display input unit 71 of the transportation controller 7 are supported by an arm from the back side of the carts C1, C4, respectively. The barcode reader 52 is connected to the main body 50 with a sufficiently long cable, and is placed near the lower side of the display input unit 51 so as to be easily taken out by the operator.

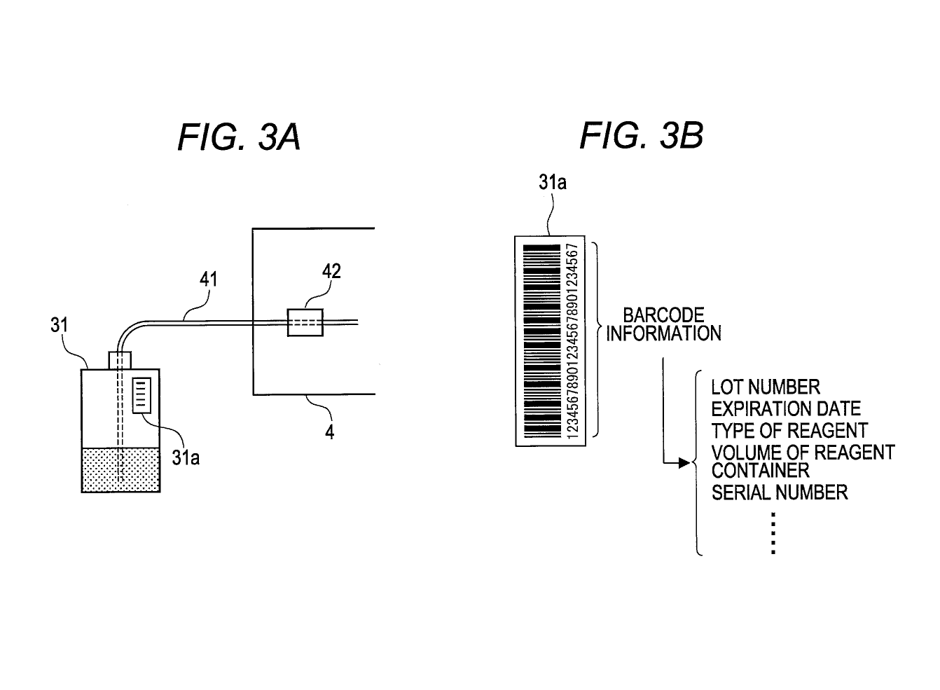

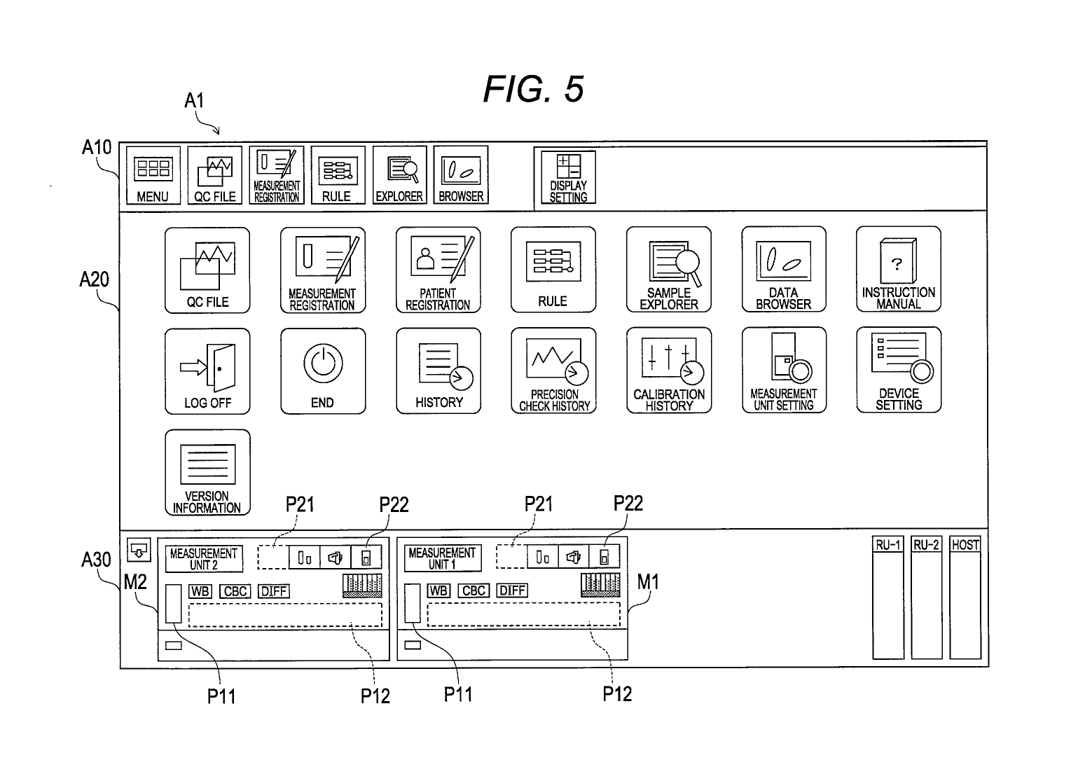

Reagent containers 31 to 36 are accommodated at the lower part of the two carts C3. The two measurement units 4 are respectively connected to the reagent containers 31 to 36 accommodated in the carts C3 through a tube 41 (see FIG. 3A), and perform measurement using the reagents. Each reagent container 31 to 36 is a container that directly accommodates a predetermined reagent, to be described later. Each reagent container 31 to 36 is attached with a barcode label 31a to 36a. When replacing the reagent, the operator reads the barcode label attached to the new reagent container with the barcode reader 52, opens the door at the front surface of the cart C3 to take out the reagent container accommodated in the cart C3, and accommodates the new reagent container in the cart C3. The replacement of the reagent will be described later with reference to FIG. 5.

FIG. 3A is a view showing a state in which the reagent container 31 is connected to the measurement unit 4.

The distal end of the tube 41 connected to the reagent container 31 is retrieved to the vicinity of the bottom surface of the reagent container 31. A prism sensor 42 is installed in the tube 41 at inside the measurement unit 4. The prism sensor 42 is configured to detect bubbles contained in the tube 41. When the prism sensor 42 detects the bubbles, determination is made that the reagent in the reagent container 31 are less than or equal to a predetermined amount. When replacing the reagent container 31, the tube 41 is detached from the reagent container 31, and then the tube 41 is connected to the new reagent container 31.

In FIG. 3A, only the reagent container 31 is shown for the sake of convenience, but the measurement unit 4 includes five tubes 41 and five prism sensors 42 corresponding to the reagent containers 32 to 36, and the reagent containers 32 to 36 are similarly connected to the measurement unit 4.

FIG. 3B is a view showing a configuration of a barcode label 31a.

Barcode information including numbers is stored in the barcode of the barcode label 31a, and numbers corresponding to the barcode information are also described on the barcode label 31a. When the barcode information of the barcode label 31a is read by the barcode reader 52, such barcode information is output to the main body 50 of the information processing unit 5 to which the barcode reader 52 is connected. The barcode information includes lot number of the reagent, expiration date of the reagent, type of reagent, volume of the reagent container, serial number, and the like. The information processing unit 5 can retrieve such information contained in the barcode information from the received barcode information.

The barcode labels 32a to 36a also have configurations similar to the barcode label 31a. In other words, the barcode information are stored in the barcode of each barcode label 32a to 36a, and numbers corresponding to the barcode information are also described on the barcode label 32a to 36a. The barcode information of the barcode labels 32a to 36a also includes lot number of the reagent, expiration date of the reagent, type of reagent, volume of the reagent container, serial number, and the like.

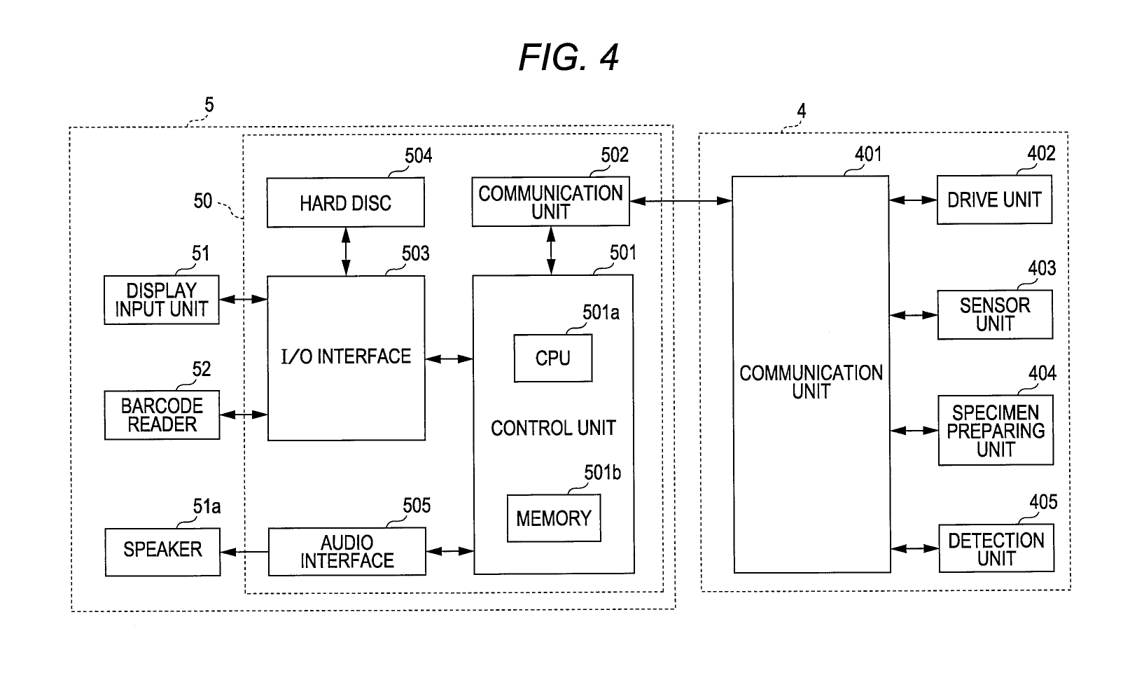

FIG. 4 is a view showing a configuration of the measurement unit 4 and the information processing unit 5. Only one measurement unit 4 is shown in FIG. 4 for the sake of convenience, but other measurement units 4 are also similarly configured.

The measurement unit 4 includes a communication unit 401, a drive unit 402, a sensor unit 403, a specimen preparing unit 404, and a detection unit 405.

The communication unit 401 is communicably connected to a communication unit 502 of the information processing unit 5. The communication unit 401 transmits a signal output from each section of the measurement unit 4 to the information processing unit 5, and outputs a signal from the information processing unit 5 to each section of the measurement unit 4. The drive unit 402 includes a mechanism for carrying out the measurement of the sample, and the sensor unit 403 includes a sensor for detecting the position of the mechanism in the measurement unit 4 and the position of the sample container, and the like. The sensor unit 403 includes the prism sensor 42 (see FIG. 3A) installed in the tube 41, to which the reagent containers 31 to 36 are respectively connected.

The specimen preparing unit 404 appropriately mixes and stirs the aspirated sample and the reagent aspirated from the reagent containers 31 to 36 in the measurement unit 4 to prepare the specimen for measurement. The detection unit 405 measures the specimen prepared by the specimen preparing unit 404. The data of the sample obtained by such measurement is transmitted to the information processing unit 5 through the communication unit 401.

As shown in FIG. 2, the information processing unit 5 includes the main body 50, the display input unit 51, the barcode reader 52, and the speaker 51a. The main body 50 includes a control unit 501, a communication unit 502, an I/O interface 503, a hard disc 504, and an audio interface 505.

The control unit 501 includes a CPU 501a and a memory 501b, and controls each unit of the information processing unit 5 by executing a computer program stored in the hard disc 504. A signal output from each unit of the information processing unit 5 is input to the control unit 501. The communication unit 502 is communicably connected to the communication unit 401 of the two measurement units 4. The communication unit 502 transmits the signal input from the control unit 501 to the two measurement units 4, and outputs the signal received from the two measurement units 4 to the control unit 501.

The control unit 501, the hard disc 504, the display input unit 51, and the barcode reader 52 are connected to the I/O interface 503. The control unit 501 reads the data from the hard disc 504 and writes the data to the hard disc 504 through the I/O interface 503. The control unit 501 also outputs a video signal to the display input unit 51 and outputs a signal to the barcode reader 52 through the I/O interface 503. The content input by the operator through the display input unit 51 and the barcode information read by the barcode reader 52 are input to the control unit 501 through the I/O interface 503.

The hard disc 504 is installed with various computer programs to be executed by the control unit 501 such as operating system and application program, as well as data used in executing the computer program. For example, the hard disc 504 is installed with a program for analyzing the data of a sample received from the two measurement units 4 to generate the measurement result such as the number of red blood cells and the number of white blood cells, and causing the display input unit 51 to perform the display based on the generated measurement result, a program for accepting the reagent replacement, and the like.

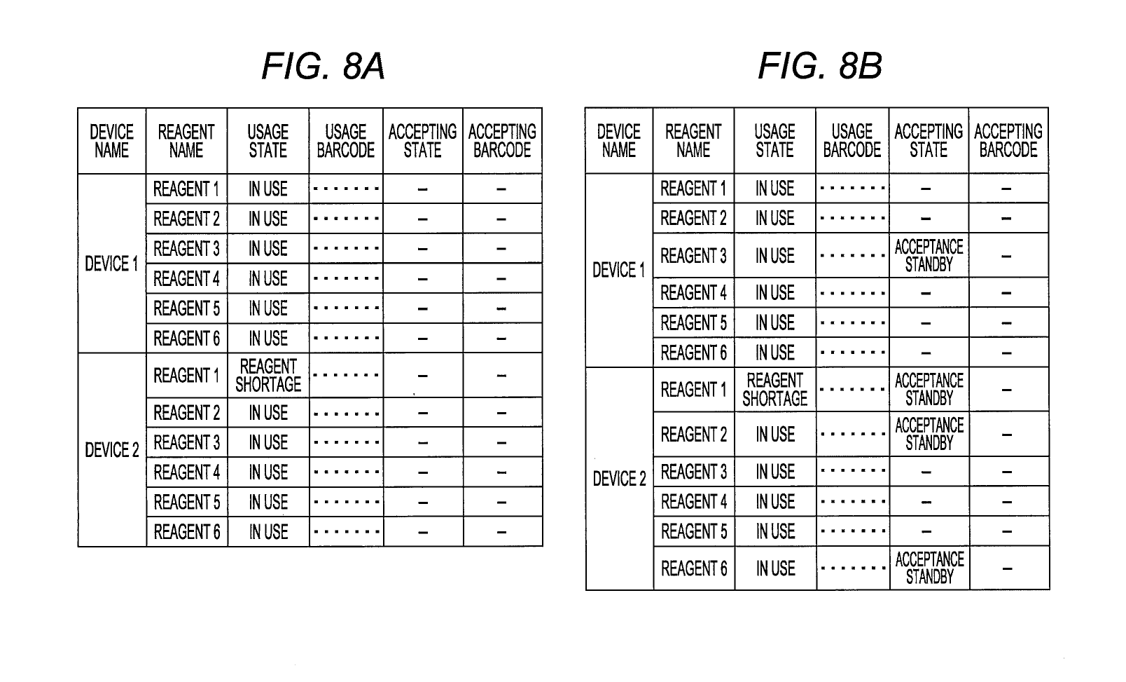

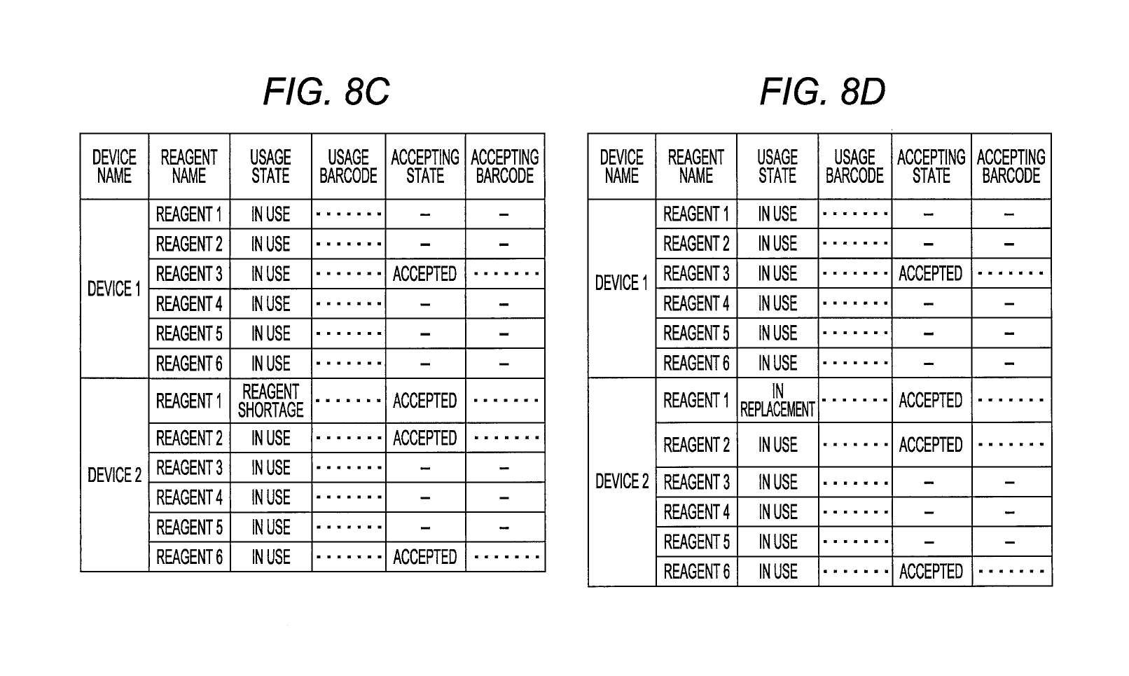

The hard disc 504 stores a “reagent management DB (database)” in which information regarding the reagent containers 31 to 36 connected to the two measurement units 4 are stored. The reagent management DB stores the information on the reagent container, which is being used (connected) in the measurement unit 4, and the like for every measurement unit 4. The reagent management DB will be described later with reference to FIGS. 8A to 8D.

The hard disc 504 stores the remaining number of usage times of the reagent containers in correspondence with the reagent container being used stored in the reagent management DB. Such number of usage times is calculated and updated for every measurement based on the capacity of the reagent container obtained from the barcode information of the reagent container, the number of times used in the measurement, and the capacity used in one measurement.

The audio interface 505 includes an audio output terminal. The audio interface 505 converts the audio signal (digital) input from the control unit 501 and outputs the audio signal (analog) from the audio output terminal. The audio output terminal of the audio interface 505 is connected to the speaker 51a by way of an analog signal cable. The speaker 51a outputs the audio signal (analog) input from the audio interface 505 as an audio.

FIG. 5 is a view showing a menu screen A1 of the information processing unit 5 displayed on the display input unit 51. FIG. 5 shows an initial state of the menu screen A1.

The menu screen A1 includes a tool bar region A10, a main region A20, and a measurement operation region A30. The tool bar region A10 and the main region A20 include a plurality of buttons. The operator can give various instructions to the information processing unit 5 by touching the buttons.

The measurement operation region A30 includes operating portions M1, M2 corresponding to the measurement unit 4 on the right side and the measurement unit 4 on the left side, respectively. The operating portions M1, M2 are similarly configured, and respectively includes a status notifying part P11, an error message display region P12, an error button P21 including an icon indicating error warning, and an operating portion menu button P22.

The status notifying part P11 is displayed in green if the corresponding measurement unit 4 is operating normally, and is displayed in red if the corresponding measurement unit 4 has error. An error message is displayed in the error message display region P12 when error occurs in the corresponding measurement unit 4. In FIG. 5, the status notifying part P11 is displayed in green, and the error message is not displayed in the error message display region P12 since there is no error.

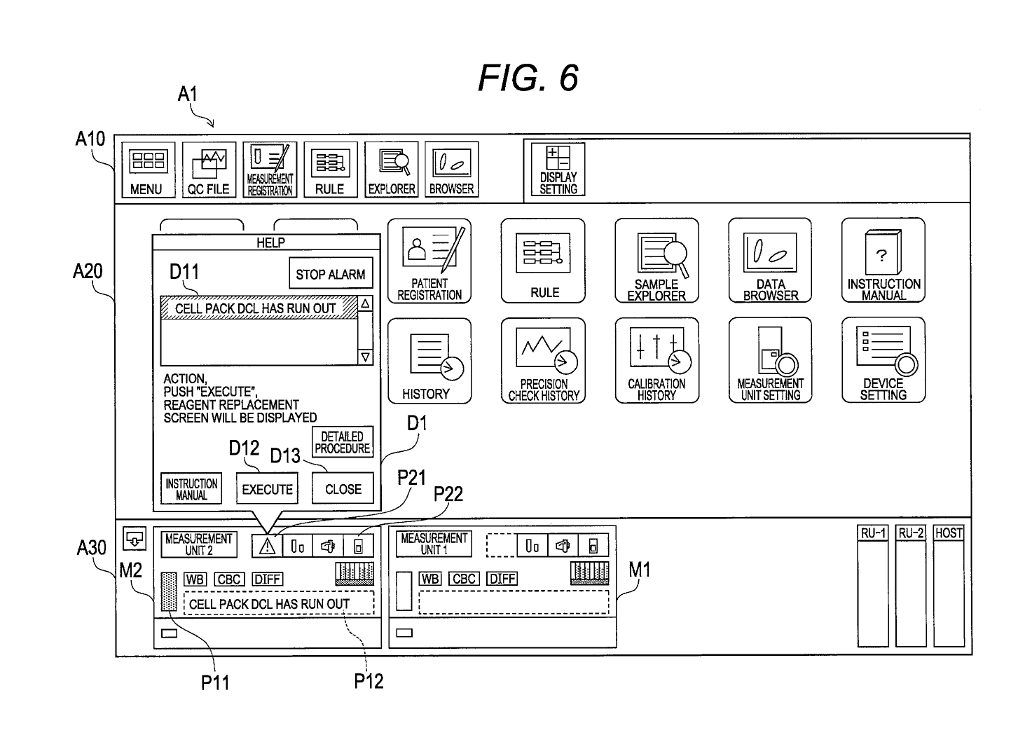

The error button P21 is a button displayed when error occurs in the corresponding measurement unit 4. When error occurs, the error button P21 is displayed, and a help dialogue (see FIG. 6) is displayed at the upper part of the corresponding operating portion. In FIG. 5, no error button P21 is displayed since there is no error in either measurement unit 4. The operating portion menu button P22 is a button for opening an operating portion menu screen (not shown) in which various processes can be specified.

FIG. 6 is a view showing the menu screen A1 in a state the help dialogue D1 is opened. FIG. 6 shows a state in which the reagent (cell pack DCL) is insufficient in the measurement unit 4 on the left side.

The color of the status notifying part P11 of the operating portion M2 is red since error occurred in the measurement unit 4 on the left side. The error message is displayed in the error message display region P12. Since the error occurred in the measurement unit 4 on the left side, the error button P21 is displayed in the operating portion M2, and the help dialogue D1 is displayed at the upper part of the operating portion M2. If the reagent runs out in the measurement unit 4 on the right side, similar help dialogue D1 is displayed at the upper part of the operating portion M1.

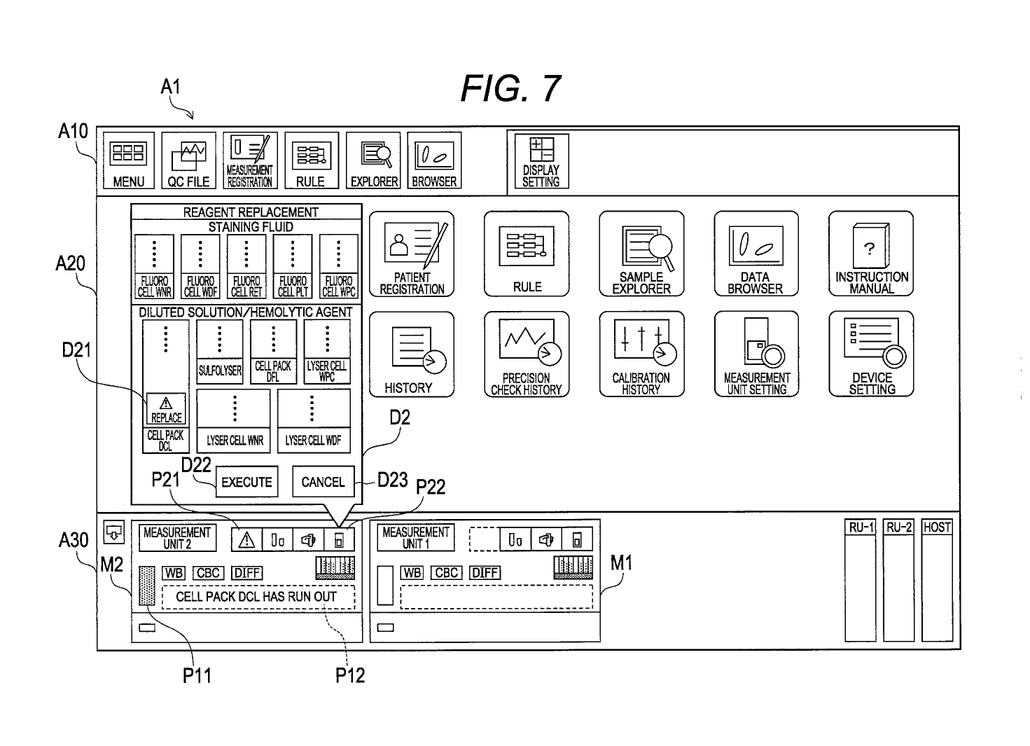

The help dialogue D1 includes an error message D11, an execute button D12, and a close button D13. The error message list D11 displays an error content, and displays a plurality of error items when a plurality of errors simultaneously occur in the measurement unit 4 on the left side. As shown in FIG. 6, when error occurs due to reagent insufficiency, the operator touches the execute button D12 to display a reagent replacement dialogue D2 (see FIG. 7) for replacing the reagent of the measurement unit 4 on the left side, which is the origin of occurrence of error.

When the operator touches the execute button D12, the help dialogue D1 is closed, and instead, the reagent replacement dialogue D2 (see FIG. 7) is displayed. When the reagent insufficiency occurs in the measurement unit 4 on the right side, and the execute button D12 of the help dialogue D1 corresponding to the measurement unit 4 on the right side is touched, similar reagent replacement dialogue D2 is displayed at the upper part of the operating portion M1. When the operator touches the close button D13 and the help dialogue D1 is closed, a state shown in FIG. 5 is obtained. If the error of the measurement unit 4 on the left side is not resolved, a state in which the error button P21 of the operating portion M2 is displayed is maintained in FIG. 5.

FIG. 7 is a view showing a menu screen A1 in a state the reagent replacement dialogue D2 is opened.

The reagent replacement dialogue D2 includes an execute button D22 and a cancel button D23. The reagent replacement dialogue D2 displays a list of reagents set in the measurement unit 4, and information indicating the reagent remaining amount of the reagent container, the expiration date of the reagent, and the like are displayed in a display region corresponding to each reagent. The “cell pack DCL”, the “sulfolyser”, “cell pack DFL”, “lyser cell WPC”, “lyser cell WNR”, and “lyser cell WDF” shown in the reagent replacement dialogue D2 are respectively corresponded to the reagent contained in the reagent containers 31 to 36 shown in FIG. 2. The reagents contained in the reagent containers 31 to 36 are respectively referred to as reagent 1 to reagent 6, for the sake of convenience.

FIG. 7 displays a state region D21 in the display region corresponding to the cell pack DCL (reagent 1). The state region D21 displays an icon indicating warning, and “replace” which indicates that replacement of the reagent container is necessary. Thus, the operator can check the information of each reagent and can check whether the reagent container needs to be replaced by checking the display region corresponding to each reagent.

When the operator touches the execute button D22 with a new reagent container prepared in place of the reagent container that needs replacement, the reagent replacing operation is started for all the reagent containers in which the replacement of the reagent is ready of the reagent containers 31 to 36. When the operator touches the cancel button D23, the reagent replacement dialogue D2 is closed, and the state shown in FIG. 5 is obtained. If the reagent replacement of the measurement unit 4 on the left side is not completed, a state in which the error button P21 of the operating portion M2 is displayed is maintained in FIG. 5.

In the state of FIG. 5, even when the expiration date of the reagent is passed, the help dialogue D1 is displayed at the upper part of the corresponding operating portion as shown in FIG. 6. In this case, the name of the reagent which expiration date passed and the passing of the expiration date are displayed in the error message list D11 and the error message display region P12. When the execute button D12 of the help dialogue D1 is touched, the reagent replacement dialogue D2 is displayed as shown in FIG. 7, and the state region D21 is displayed in the display region of the corresponding reagent, similar to the case of the reagent insufficiency.

The reagent replacement dialogue D2 is not only displayed by the execute button D12 of the help dialogue D1, and is also displayed even if the reagent insufficiency or the passing of the expiration date of the reagent has not occurred. In other words, even if the measurement unit 4 does not have error and the menu screen A1 is displayed as shown in FIG. 5, the operator can display the reagent replacement dialogue D2 through the operating portion menu screen (not shown) displayed by touching the operating portion menu button P22.

If the reagent replacement dialogue D2 is displayed when there is no error, the state region D21 is not displayed in the reagent replacement dialogue D2. In this case, the operator can check the display region corresponding to each reagent of the reagent replacement dialogue D2, and replace in advance the reagent in which the reagent remaining amount is small and the reagent which expiration date is soon.

A case in which the reagent replacement task is carried out using the reagent replacement dialogue D2 will be described below. The procedure of a case of replacing the reagent container 31 of the cell pack DCL (reagent 1) will be described. The procedure for replacing the reagent containers 32 to 36 is similar to the procedure for replacing the reagent container 31, and thus the description will be omitted herein.

The operator first references the reagent replacement dialogue D2 displayed as shown in FIG. 7 on the display input unit 51 (see FIG. 2), and confirms that the reagent container 31 of the cell pack DCL (reagent 1) needs to be replaced. The operator then uses the barcode reader 52 (see FIG. 2) to read the barcode information from the barcode label 31a attached to the new reagent container 31. In this case, the operator references the display region corresponding to the reagent 1 of the reagent replacement dialogue D2, and checks whether or not the new reagent container 31 can be appropriately used from the reagent remaining amount, expiration date, and the like of the new reagent container 31. The operator thereafter detaches the tube 41 (see FIG. 3A) connected to the reagent container 31 that needs reagent replacement, and reconnects the tube 41 to the new reagent container 31 which barcode information is read.

The operator touches the execute button D22 displayed on the display input unit 51 to start the reagent replacing operation of the reagent container 31. The reagent 1 is thereby aspirated from the new reagent container 31 through the tube 41, and whether or not the reagent container 31 is appropriately connected is determined from the output signal of the prism sensor 42. The new reagent container 31 thus becomes a usable state, and the reagent replacing operation of the reagent container 31 is completed.

However, if the reagent replacement task is carried out using the reagent replacement dialogue D2, the operator needs to go back and forth between the installed position of the reagent container and the display input unit 51, and carry out a series of tasks such as reading the barcode information from the reagent container with the barcode reader 52, attaching/detaching the tube 41 with respect to the reagent container, and input (touch operation) to the display input unit 51.

Specifically, if the new reagent containers 31 to 36 are arranged in the vicinity of the installed position of the reagent container (e.g., back side of the cart C3) in advance, the operator needs to read the barcode information of the new reagent at the relevant position, and move to the front of the display input unit 51 from the vicinity of the installed position to check the information such as the reagent remaining amount and the expiration date of the reagent container in which reading is carried out. After reconnecting the tube 41 to the new reagent container, the operator also needs to move to the front of the display input unit 51 from the vicinity of the cart C3 and touch the execute button D22 displayed on the display input unit 51 to start the reagent replacing operation for the relevant reagent container. Thus, the operator needs to go back and forth between the installed position of the reagent container 31 to 36 and the display input unit 51 for every reagent replacement.

In the present embodiment, the reagent replacement mode by the audio guidance is prepared separate from the reagent replacement mode using the reagent replacement dialogue D2 described above. In the reagent replacement mode by the audio guidance, the reference of the display input unit 51 can be omitted and the operation to the display input unit 51 can be omitted by the reagent management DB configured as described below, the reading of the barcode reader 52, and the audio output from the speaker 51a. The operator thus can carry out the reagent replacement similar to the above without going back and forth between the installed position of the reagent container 31 to 36 and the display input unit 51.

In the reagent replacement mode by the audio guidance, the barcode label of the (existing) reagent container currently being connected to the measurement unit 4 is read by the barcode reader 52, so that the relevant reagent container is set as the target of replacement. The operator reads the barcode label of the desired reagent container of the existing reagent containers with the barcode reader 52, so that the relevant reagent container can be set as the target of replacement. After the barcode label of the (existing) reagent container currently being used is read, the audio guidance indicating the operation necessary for replacing the relevant reagent container with a new reagent container is output from the speaker 51a. This audio guidance includes the content that urges the replacement to a new reagent container and the content that urges the barcode label of the new reagent container to be read after the preparation for replacement is completed. The audio guidance includes content indicating the status (remaining amount, etc.) of the reagent in the existing reagent container set to be the target of replacement. The operator replaces the existing reagent container with the new reagent container according to the audio guidance, and then reads the barcode label of the new reagent container with the barcode reader 52 to replace with the new reagent container. After the operator carries out the operation necessary for replacing to a new reagent container, the reagent information of the new reagent container is registered in the reagent management DB, and the operation (reagent replacing operation) of aspirating the reagent contained in the new reagent container is executed in the measurement unit 4. The replacement of the reagent is thereby completed.

The reagent management DB is prepared and a control using the reagent management DB is carried out to realize the reagent replacement mode described above. A configuration of the reagent management DB and a flowchart showing the flow of control will be hereinafter described.

FIGS. 8A to 8D are conceptual diagrams showing a configuration of the reagent management DB.

The reagent management DB includes a device name item, a reagent name item, a usage state item, a usage barcode item, an accepting state item, and an accepting barcode item. In the device name item, “device 1”, which represents the measurement unit 4 on the right side, or “device 2”, which represents the measurement unit 4 on the left side is set. The measurement unit 4 on the right side and the measurement unit 4 on the left side are hereinafter respectively referred to as “device 1” and “device 2”.

In the reagent name item, the type (name) of the reagent container connected to the devices 1, 2 is set. In the present embodiment, the type of reagent container connected to the devices 1, 2 is reagents 1 to 6, as described above.

In the usage state item, the usage state of the reagent container connected to the devices 1, 2 is set. Specifically, in the usage state item, one of “in use”, which indicates that the reagent container is being used without any problem, “reagent shortage”, which indicates that the relevant reagent container is short of reagent, “past expiration date” which indicates that the reagent container passed the expiration date, and “in replacement”, which indicates that the reagent replacing operation is being carried out for the relevant reagent container is set. In the usage barcode item, the barcode information of the reagent container connected to the devices 1, 2 is set.

In the accepting state item, when replacing the reagent container connected to the device 1, 2, one of “−”, which indicates no-setting state (Reset state), “acceptance standby”, and “accepted” is set according to the progress of acceptance of the new reagent container. The “acceptance standby” state is a state in which the existing reagent currently connected to the measurement unit 4 is set as the target of replacement, and the “accepted” state is a state in which the new reagent is accepted for replacement with respect to the existing reagent set as the target. The accepting barcode item is set with either “−”, which indicates the no-setting state (reset state), or the barcode information attached to the new reagent container.

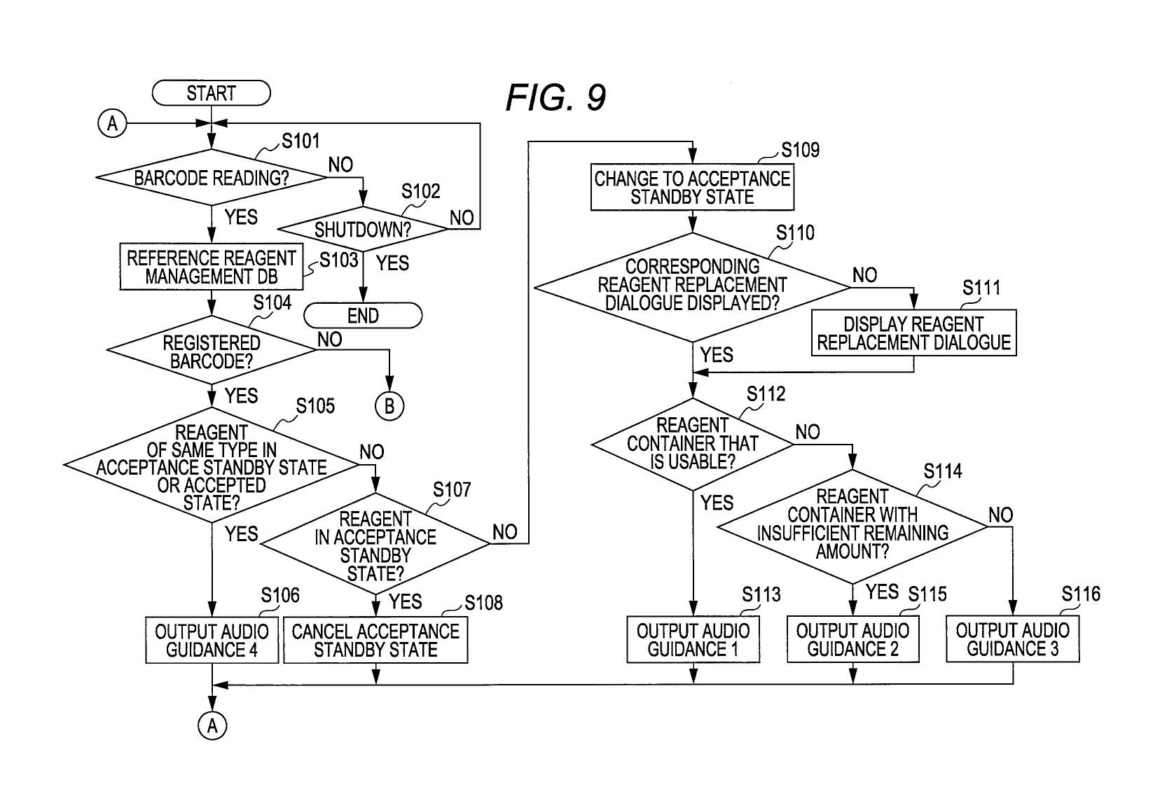

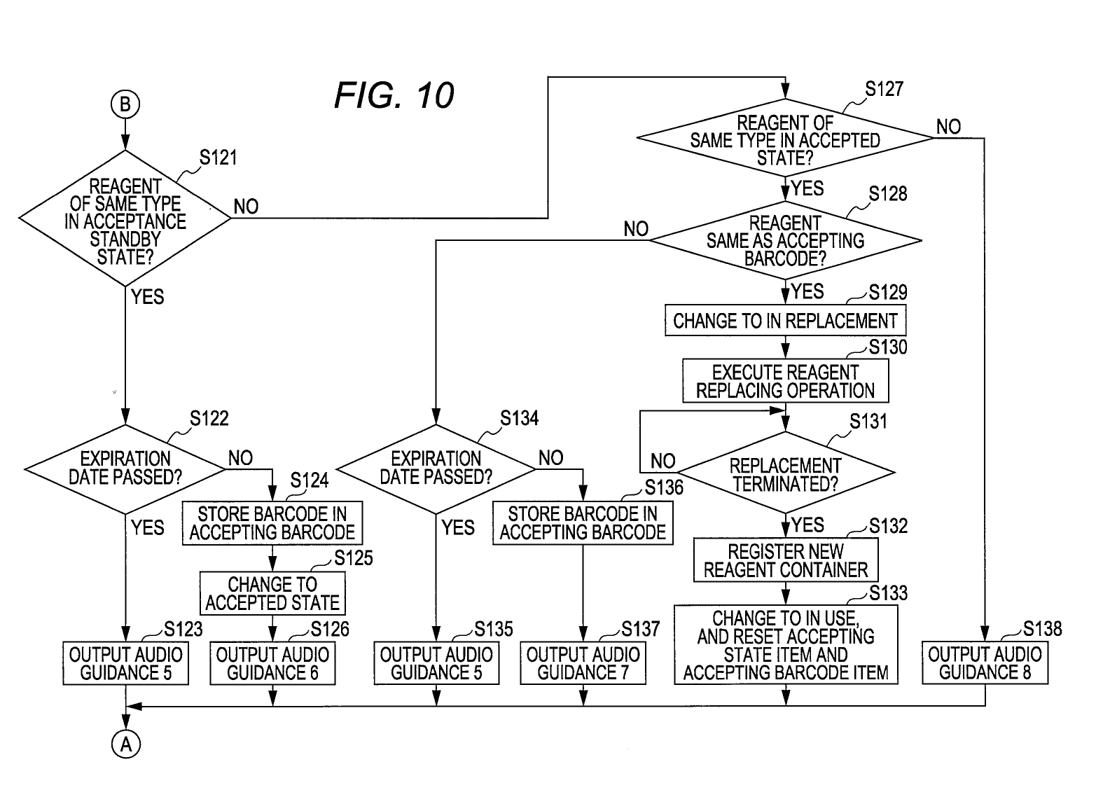

FIGS. 9 and 10 are flowcharts showing processes performed by the information processing unit 5. The flowchart shown in FIGS. 9 and 10 is started at the startup of the information processing unit 5, and is executed regardless of the display content of the display input unit 51 (regardless of presence or absence of error such as reagent shortage and past expiration date of the reagent.

With reference to FIG. 9, the control unit 501 of the information processing unit 5 waits for the process until the barcode information is read by the barcode reader 52 (S101). In other words, if the barcode information is not read by the barcode reader 52 (S101: NO), the process returns to S101 unless shutdown is carried out (S102: NO).

After the barcode information is read by the barcode reader 52 (S101: YES), the control unit 501 references the reagent management DB stored in the hard disc 504 (S103). The control unit 501 then determines whether or not the barcode information read in S101 exists in the usage barcode item of the reagent management DB (S104). In other words, whether or not the reagent container having the barcode information read in S101 is the reagent container (hereinafter referred to as “existing reagent container”) currently connected to one of the devices 1, 2 is determined.

If the barcode information read in S101 is not the barcode information of the existing reagent container (S104: NO), the process proceeds to a combinator B (S121 of FIG. 10). If the barcode information read in S101 is the barcode information of the existing reagent container (S104: YES), the control unit 501 determines whether or not the accepting state item is in the “acceptance standby” state or the “accepted” state for the reagent of the same type as the type of reagent indicated by the barcode information (S105). For instance, when the reagent management DB is in a state shown in FIG. 8A, and the barcode information read in S101 is the barcode information of the reagent 1 connected to the device 2 (when matching the usage barcode item of the reagent 1 of the device 2), the accepting state item of the reagent of the same type as the relevant reagent, that is, the reagent 1 of the device 1 is in the “−” state, and thus determination is made as NO in S105. A case in which determination is made as YES in S105 will be described later.

If determined as NO in S105, the control unit 501 determines whether or not the accepting state item of the reagent indicated by the barcode information read in S101 is the “acceptance standby” state (S107). For instance, when the reagent management DB is in a state shown in FIG. 8A, and the barcode information read in S101 is the barcode information of the reagent 1 connected to the device 2, the accepting state item of the reagent of is “−”, and thus determination is made as NO in S107. A case in which determination is made as YES in S107 will be described later.

If determined as NO in S107, the control unit 501 changes the accepting state item of the reagent indicated by the barcode information read in S101 to the “acceptance standby” state (S109). The control unit 501 then determines whether the corresponding reagent replacement dialogue D2 is displayed on the display control unit 51 (S110). If the corresponding reagent replacement dialogue D2 is not displayed (S110: NO), the control unit 501 displays the corresponding reagent replacement dialogue D2 on the display input unit 51 (S111).

For example, when the display of the display input unit 51 is in the state shown in FIG. 7, if the reagent indicated by the barcode information read in S101 is the reagent of the device 2, the corresponding reagent replacement dialogue D2 is already displayed, and thus determination is made as YES in S110. If the reagent indicated by the barcode information read in S101 is the reagent of the device 1 in this case, the corresponding reagent replacement dialogue D2 is not displayed at the upper part of the operating portion M1, and thus determination is made as NO in S110 and the reagent replacement dialogue D2 of the device 1 is displayed.

The control unit 501 then determines whether the existing reagent container having the barcode information read in S101 is useable, has insufficient remaining amount, or past the expiration date (S112, S114), and outputs the corresponding audio guidance from the speaker 51a.

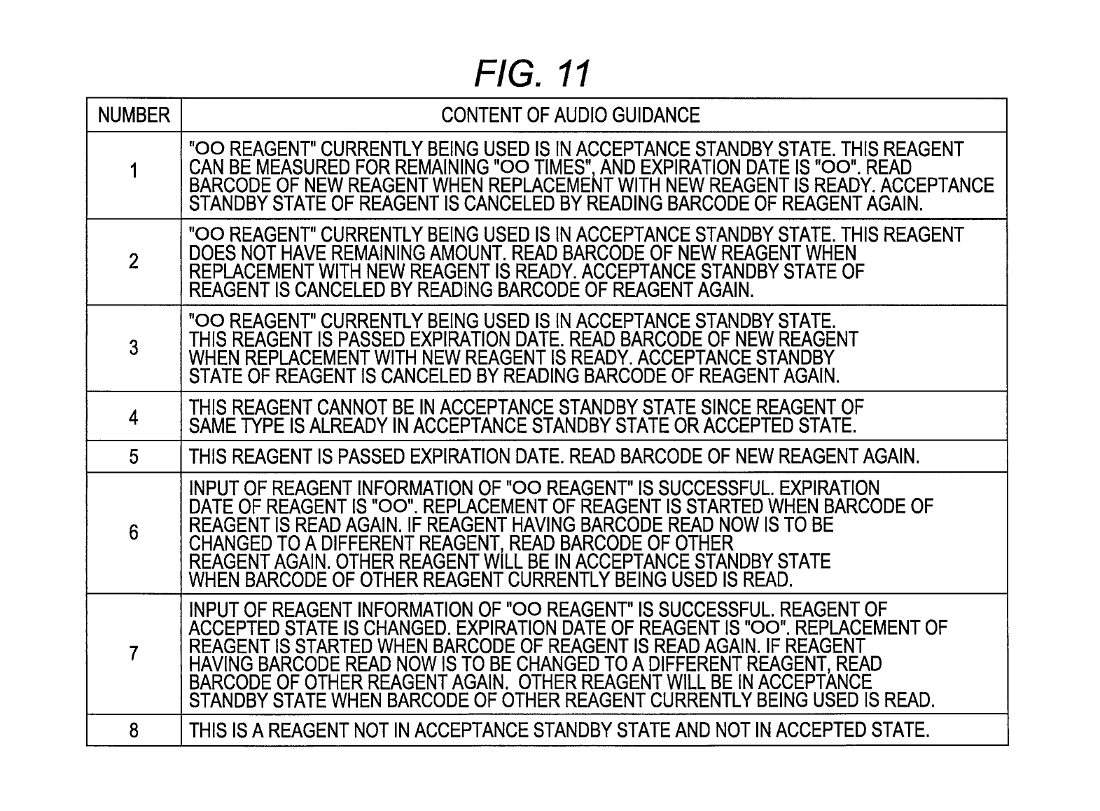

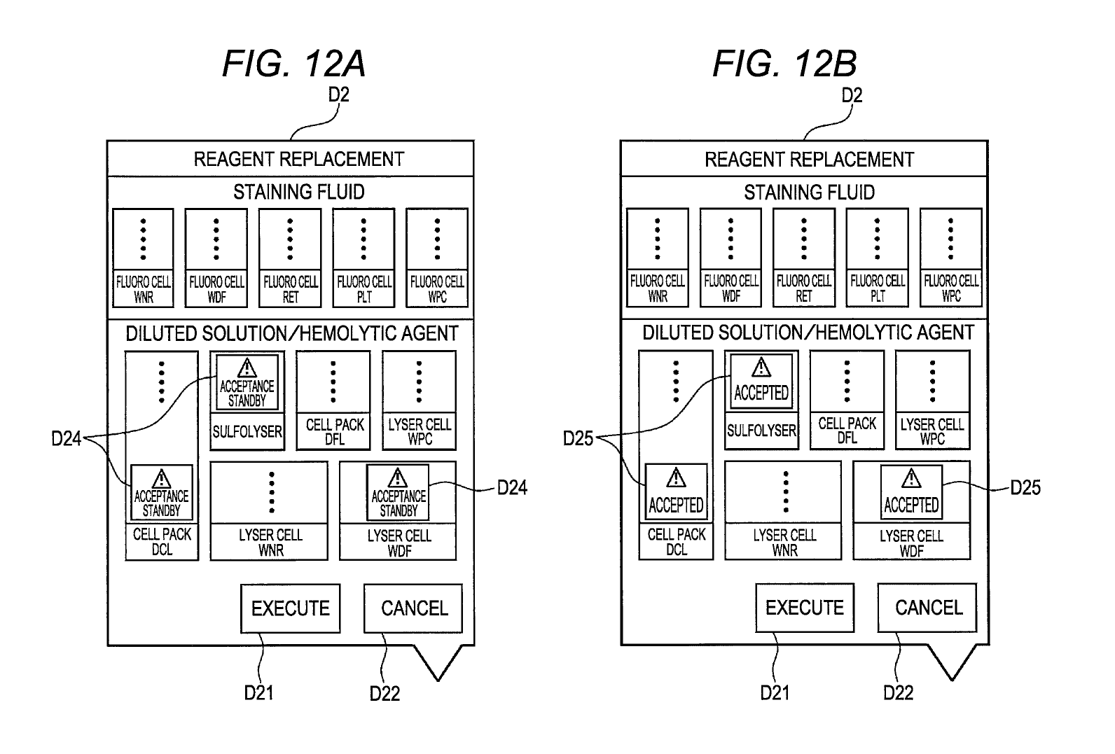

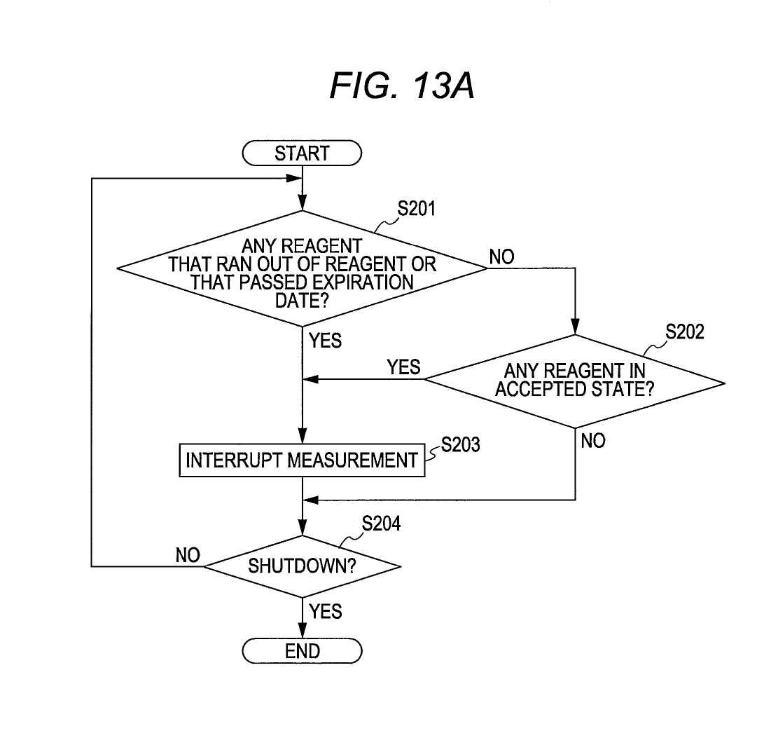

If the existing reagent container is useable, that is, is not running out of reagent and is not past the expiration date (S112: YES), the audio guidance 1 indicated with number 1 in FIG. 11 is output (S113). If the existing reagent container is running out of remaining amount (S112: NO, S114: YES), the audio guidance 2 indicated with number 2 in FIG. 11 is output (S115). If the existing reagent container is past the expiration date (S112: NO, S114: NO), the audio guidance 3 indicated with number 3 in FIG. 11 is output (S116). According to such audio guidance, the operator can recognize that the existing reagent container is the target of replacement, the state of the reagent contained in the existing reagent container, the replacement procedure to the new reagent container, and the procedure for canceling the existing reagent container as the target of replacement.

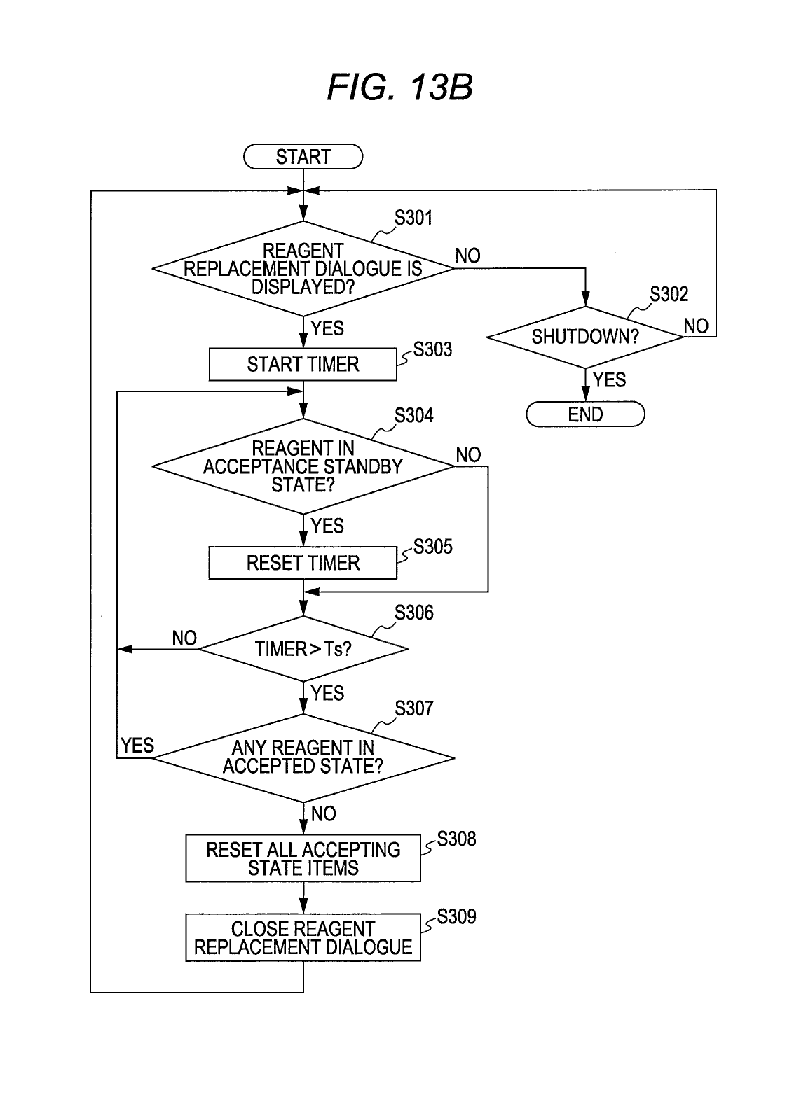

After the existing reagent container is set to the acceptance standby state, and the processes of S113, S115, S116 are finished, the process proceeds to a combinator A (S101). As shown in FIG. 12A, when the existing reagent container is set to the acceptance standby state, the state region D24 including the display “acceptance standby” indicating that the replacement of the reagent container is accepted is displayed in the display region corresponding to the existing reagent container in the reagent replacement dialogue D2. The accepting state item of the corresponding reagent of the reagent management DB is updated to “acceptance standby”. For instance, when the barcode information is continuously read for the existing reagent container of the reagent 3 of the device 1 and the existing reagent container of the reagents 1, 2, 6 of the device 2 from the state of the reagent management DB shown in FIG. 8A, the accepting state item corresponding to such reagents becomes “acceptance standby” as shown in FIG. 8B.

A case in which the barcode information is read from the existing reagent container of the reagent 1 of the device 1 when the state of the reagent management DB is the state shown in FIG. 8B will now be described. In this case, after determined as YES in S101, S104, determination is made as YES in S105 since the accepting state item of the reagent of the same type as the type of the reagent indicated by the barcode information read in S101, that is, the reagent 1 of the device 2 is in the “acceptance standby” state. If the accepting state item of the reagent 1 of the device 2 is the “accepted” state as will be described later (see FIG. 8C), determination is made as YES in S105 since the accepting state item of the reagent of the same type as the type of the reagent indicated by the barcode information read in S101, that is, the reagent 1 of the device 2 is the “accepted” state.

Therefore, since two or more reagents of the same type are not simultaneously in the acceptance standby state or the accepted state, the control unit 501 can specify to which reagent of the same type to perform the reagent replacing operation, and hence the process can be simplified. If determined as YES in S105, the control unit 501 outputs an audio guidance 4 indicated with number 4 in FIG. 11 from the speaker 51a (S106). The operator thus can recognize that the existing reagent container of the reagent 1 of the device 1 cannot be set to the target of reagent replacement.

A case in which the barcode information is read from the existing reagent container of the reagent 1 of the device 2 when the state of the reagent management DB is the state shown in FIG. 8B will now be described. In this case, after determined as YES in S101, S104 and NO in S105, determination is made as YES in S107 since the accepting state item of the reagent indicated by the barcode information read in S101, that is, the reagent 1 of the device 2 is the “acceptance standby” state.

Therefore, when the barcode information is again read from the existing reagent container that is already in the acceptance standby state (S107: YES), the control unit 501 cancels (reset state) the accepting state item of the existing reagent container (S108). The existing reagent container thereby returns to the state before the acceptance of the reagent replacement. The procedure for canceling the acceptance standby state is informed to the operator in advance in the audio guidance 1 to 3 output in S113, S115, S116.

When the state of the reagent management DB is a state shown in FIG. 8C, if the barcode information is read from the existing reagent container of the reagent 1 of the device 2, the accepting state item of the reagent 1 of the device 2 is the “accepted” state, and thus determination is made as NO in S105 and S107. In this case, the accepting state item of the reagent already in the “accepted” state is returned to the “acceptance standby” state, and the item of the accepting barcode is reset.

The process of replacing the existing reagent container set to the acceptance standby will now be described.

The operator reads the barcode information of the existing reagent container, on which to perform the reagent replacement, with the barcode reader 52, and then reads the barcode information of a new reagent container (hereinafter referred to as “new reagent container”) of the same type as the existing reagent container with the barcode reader 52. When the barcode information is read from the new reagent container (S101: YES), the control unit 501 of the information processing unit 5 references the reagent management DB (S103), and determines whether or not the read barcode information is registered in the reagent management DB (S104), similar to above. If the barcode information read in S101 is not the barcode information of the existing reagent container (S104: NO), the process proceeds to a combinator B (S121 of FIG. 10).

With reference to FIG. 10, the control unit 501 determines whether the reagent of the same type as the type of reagent indicated by the barcode information read in S101 is in the acceptance standby state in the reagent management DB (S121). For example, when the reagent management DB is in the state shown in FIG. 8B, if the type of reagent indicated by the barcode information read in S101 is “reagent 1”, determination is made as YES in S121 since the reagent 1 of the device 2 is in the “acceptance standby” state.

If determined as YES in S121, the control unit 501 determines whether or not the new reagent container passed the expiration date based on the expiration date indicated by the barcode information read in S101 (S122). If the new reagent container passed the expiration date (S122: YES), the control unit 501 outputs the audio guidance 5 indicated with number 5 in FIG. 11 from the speaker 51a (S123). The operator can recognize that the new reagent container is not suited for replacement by the warning related to the expiration date contained in the audio guidance 5. If the new reagent container has not passed the expiration date (S122: NO), the control unit 501 stores the barcode information in the corresponding accepting barcode item of the reagent management DB (S124), and changes the accepting state item of the corresponding reagent to the “accepted” state (S125).

For example, when the reagent management DB is in the state shown in FIG. 8B, if the type of reagent indicated by the barcode information read in S101 is “reagent 1”, the barcode information read in S101 is stored in the accepting barcode item corresponding to the reagent 1 of the device 2, and the accepting state item corresponding to the reagent 1 of the device 2 becomes the “accepted” state. Similarly, with respect to the new reagent containers of the reagents 2, 3, and 6, when the barcode information is successively read, as shown in FIG. 8C, the read barcode information of the new reagent container is stored in the corresponding accepting barcode item, and the accepting state item corresponding to the read reagent becomes the “accepted” state.

As shown in FIG. 12B, when the existing reagent container is set from the acceptance standby state to the accepted state, the state region D25 including the display “accepted” indicating that the new reagent container is accepted is displayed in the display region corresponding to the existing reagent container in the reagent replacement dialogue D2.

The control unit 501 outputs an audio guidance 6 indicated with number 6 in FIG. 11 from the speaker 51a (S126). The operator thus can recognize that the replacement operation on the new reagent container is accepted, the state of the reagent contained in the new reagent container, the replacement procedure to the new reagent container, and the procedure for setting the other existing reagent container as the target of replacement. Thereafter, the process returns to the combinator A (S101 of FIG. 9), and the process is waited until the barcode information is again read. If found that the new reagent container can be used in S126, the operator detaches the tube 41 shown in FIG. 3A from the existing reagent container (existing reagent container set as the target of replacement) of the same type as the new reagent container, and connects it to the new reagent container.

When the barcode information is read from the new reagent container and the process proceeds from S101, S103, and S104 of FIG. 9 to S121 of FIG. 10, if determined as NO in S121, the control unit 501 determines whether the reagent of the same type as the type of reagent indicated by the barcode information read in S101 is in the accepted state in the reagent management DB (S127). For example, when the reagent management DB is in the state shown in FIG. 8C, if the type of reagent indicated by the barcode information read in S101 is “reagent 1”, determination is made as YES in S127 since the reagent 1 of the device 2 is in the “accepted” state.

If determined as YES in S127, the control unit 501 determines whether the barcode information read in S101 is stored in the accepting barcode item corresponding to the reagent determined as the accepted state in S127 (S128). For example, when the reagent management DB is in the state shown in FIG. 8C, determination is made as YES in S128 if the barcode information read in S101 matches the content of the accepting barcode item corresponding to the reagent 1 of the device 2. In other words, determination is made as YES in S128 if the barcode information is read twice consecutively with respect to the same new reagent container, and determination is made as NO in S128 if the new reagent container from which the barcode information is read this time is different from the new reagent container from which the barcode information is read the previous time.

If determined as YES in S128, the control unit 501 changes the usage state item corresponding to the new reagent container to “in replacement” (S129) as shown with the reagent 1 of the device 2 of FIG. 8D, and executes the reagent replacing operation on the new reagent container connected to the measurement unit 4 (S130). Specifically, the reagent is aspirated from the new reagent container through the tube 41, and determination is made whether the remaining amount of the reagent in the reagent container is sufficient, and the like by a prism sensor 42.

After the reagent replacing operation is terminated (S131: YES), the control unit 501 registers the new reagent container in the reagent management DB (S132). The control unit 501 changes the usage state item corresponding to the reagent performed with the reagent replacing operation to “in use”, and resets the accepting state item and the accepting barcode item corresponding to the reagent performed with the reagent replacing operation (S133). For example, if the reagent replacing operation is executed on the reagent 1 of the device 2, as shown in FIG. 8D, when the reagent replacing operation is terminated, the content of the accepting barcode item corresponding to the reagent 1 of the device 2 is stored in the usage barcode item corresponding to the reagent 1 of the device 2. Furthermore, the usage state item corresponding to the reagent 1 of the device 2 is changed to “in use”, and the accepting state item and the accepting barcode item become “−”. The replacement of the reagent is terminated in such manner.

A case in which the barcode information of another new reagent container is read when the reagent management DB is in the state shown in FIG. 8C will now be described.

If the reagent of the new reagent container in which the barcode information is read in S101 is the reagent of the same type as one of the existing reagents 1, 2, 3, 6, and the barcode information of the new reagent container is different from the barcode information already stored in the accepting barcode item corresponding to the existing reagent of the same type, determination is made as NO in S121, YES in S127, and NO in S128. In this case, the control unit 501 determines whether or not the new reagent container passed the expiration date from the barcode information read in S101 (S134). If the new reagent container passed the expiration date (S134: YES), the control unit 501 outputs the audio guidance 5 indicated with number 5 in FIG. 11 from the speaker 51a (S135).

If the new reagent container has not passed the expiration date (S134: NO), the control unit 501 switches to the barcode information already stored in the accepting barcode item of the reagent 1 of the device 2 to store the barcode information read in S101 (S136), and outputs an audio guidance 7 indicated with number 7 in FIG. 11 from the speaker 51a (S137). The operator thus can recognize the content similar to the audio guidance 6 and that the new reagent container, which is the target of replacement, is changed.

When the barcode information is read from the new reagent container, if the existing reagent container corresponding to the new reagent container is not yet set to the acceptance standby state nor the accepted state, that is, if the barcode information is read from the new reagent container before reading the barcode information from the existing reagent container, determination is made as NO in S127, and the control unit 501 outputs an audio guidance 8 indicated with number 8 in FIG. 11 from the speaker 51a (S138). The operator thus can recognize that the reagent replacement procedure is not appropriate. Determination is made as NO in S127 if determined that the type of reagent contained in the reagent container is not included in the reagent name stored in the reagent management DB or if the format of the barcode information is non-conforming from the barcode information read in S101. In this case as well, the control unit 501 outputs the audio guidance 8 indicated with number 8 in FIG. 11 from the speaker 51a (S138).

FIG. 13A is a flowchart showing a measurement interruption process executed for the measurement unit 4. The measurement interruption process is executed independently on the two measurement units 4. Only a case in which the process is executed in one of the measurement units 4 will be described below for the sake of convenience.

The control unit 501 of the information processing unit 5 determines if there is a remaining amount of the reagent containers 31 to 36 based on the prism sensor 42 installed in correspondence with the tube 41 connected to the reagent containers 31 to 36, and determines if the existing reagent container connected to the measurement unit 4 is within the expiration date based on the usage barcode item of the reagent management DB (S201).

If there is an existing reagent container in which the reagent is insufficiency or the expiration date is passed (S201: YES), the control unit 501 interrupts the measurement after the sample scheduled to be measured next in the measurement unit 4 (S203). If there is no existing reagent container in which the reagent is insufficient or existing reagent container in which the expiration date passed (S201: NO), the control unit 501 references the reagent management DB and determines if there is an existing reagent container in which the accepting state item is the “accepted” state of the measurement unit 4 (S202).

If there is an existing reagent container that is in the accepted state (S202: YES), the control unit 501 interrupts the measurement of the sample scheduled to be measured next (S203), similar to S203. If there is no existing reagent container that is in the accepted state (S202: NO), the process returns to S201 unless shutdown is carried out (S204: NO).

FIG. 13B is a flowchart showing a process of closing the reagent replacement dialogue D2 executed for the measurement unit 4. The process of closing the reagent replacement dialogue D2 is executed independently for the two measurement units 4. Only a case in which the process is executed in one of the measurement units 4 will be described below for the sake of convenience.

The control unit 501 of the information processing unit 5 waits the process until the reagent replacement dialogue D2 corresponding to the measurement unit 4 is displayed on the display input unit 51 (S301). In other words, if the reagent replacement dialogue D2 corresponding to the measurement unit 4 is not displayed (S301: NO), the process returns to S301 unless the shutdown is carried out (S302: NO).

When the reagent replacement dialogue D2 corresponding to the measurement unit 4 is displayed (S301: YES), the control unit 501 starts the timer (S303), and determines whether or not which of the reagents 1 to 6 is newly in the acceptance standby state in the reagent management DB corresponding to the measurement unit 4 (S304).

When one of the reagents is in the acceptance standby state (S304: YES), the control unit 501 resets the counter of the timer started in S303 (S305). If none of the reagents is in the acceptance standby state (S304: NO), the counting of the timer is continued, and the process proceeds to S306.

The control unit 501 then determines whether or not the count of the timer started in S303 is greater than Ts (S306). If the count of the timer is greater than Ts (S306: YES), the control unit 501 determines whether there is a reagent in the accepted state in the reagent management DB corresponding to the measurement unit 4 (S307). If the count of the timer is smaller than or equal to Ts (S306: NO), and if there is a reagent in the accepted state (S307: YES), the process returns to S304.

If there is no reagent in the accepted state (S307: NO), the control unit 501 resets all accepting state items corresponding to the measurement unit 4 (S308). Thus, even if the reagent in which the accepting state item is the “acceptance standby” state, that is, the reagent set to be the target of replacement is remaining, the acceptance of reagent replacement with respect to the relevant reagent is canceled. The control unit 501 then closes the reagent replacement dialogue D2 corresponding to the measurement unit 4 (S309), and returns the process to S301.

According to the present embodiment described above, the operator first reads the barcode information of the existing reagent container to be replaced and then reads the barcode information of the new reagent container when carrying out reagent replacement. Thereafter, the operator again reads the barcode information of the new reagent container, whereby the reagent replacing operation is started on the new reagent container. The audio guidance indicated with the procedures is output from the speaker 51a according to the progress. At the time of the reagent replacing task, the operator can start the reagent replacing operation without the need to go back and forth between the installed position of the reagent container and the display input unit 51. Therefore, even if the sample processing system 1 is enlarged and the reagent containers 31 to 36 are installed at positions distant from the display input unit 51, the reagent replacing operation can be smoothly started.

According to the present embodiment, the audio guidance 1 to 3 are output when the accepting state item is the acceptance standby state, and the audio guidance 6 is output when the accepting state item is the accepted state. The operator thus can check that the existing reagent container and the new reagent container are appropriately input to the information processing unit 5 with the audio guidance without moving away from the installed position of the reagent container.

According to the present embodiment, the audio guidance 1 to 3 includes the reagent name of the existing reagent container in the acceptance standby state and the procedure for reagent replacement, and the audio guidance 6 includes the reagent name of the new reagent container in the accepted state and the procedure for reagent replacement. The operator thus can check whether the existing reagent container and the new reagent container to replace are intended reagents, and smoothly proceed the reagent replacing task. The audio guidance 1 to 3 also urges the reading of the barcode information of the new reagent container. The operator thus can proceed to the replacement to the new reagent container with a simple and easy operation of reading the barcode information of the new reagent container after the existing reagent container is set to the acceptance standby state.

According to the present embodiment, whether or not the expiration date of the new reagent information is passed is determined in S122, S134 of FIG. 10 and the audio guidance 5 is output if the expiration date is passed. The operator thus can recognize that the expiration date of the new reagent container is passed thus can avoid starting the reagent replacing operation by mistake.

According to the present embodiment, when the barcode information of the new reagent container is read again according to the audio guidance 6 after achieving the accepted state, the reagent replacing operation is started and the new reagent container is registered in the reagent management DB (S132 of FIG. 10). The operator thus can register the new reagent container in the reagent management DB by following the procedure shown by the audio guidance without moving away from the installed position of the reagent.

As shown in FIG. 13A, according to the present embodiment, the measurement after the sample scheduled to be measured next is interrupted when the reagent of reagent insufficiency and the reagent passed the usage expiration date are present, and when the accepted new reagent container is present. The operator thus can smoothly perform the task involved in the reagent replacement such as the connection of the tube 41.

According to the present embodiment, when the barcode information of the new reagent container of the same type as the type of reagent of the accepted new reagent container and different from the new reagent container is read (S128: NO in FIG. 10), the barcode information of the new reagent container is stored in place of the barcode information already stored in the accepting barcode item. In this case, the audio guidance 7 including notice that the accepted reagent is changed is output. Thus, when the period until the expiration date of the new reagent container in the accepted state is short, and the like, the operator can have a more preferable new reagent container in the accepted state instead of the relevant new reagent container. Since the audio guidance 7 includes the content similar to the audio guidance 6, the operator can proceed the reagent replacing task according to the audio guidance 7.

In this case, even if the barcode information of another new reagent container is read by mistake although there is no intention of changing the new reagent container in the accepted state to the another new reagent container, the operator can recognize the wrong reading operation, and appropriately carry out the desired tasks such as again returning the new reagent container that is originally in the acceptance standby state again to the accepted state.

According to the present embodiment, the audio guidance 1 output in S113 of FIG. 10 includes the remaining number of usage times and the expiration date of the existing reagent container from which the barcode information is read. The operator thus can appropriately determine the timing to replace the existing reagent container, and can recognize if the existing reagent container is in a state to be replaced.

According to the embodiment described above, the operator can display the reagent replacement dialogue D2 on the display input unit 51 without touching the execute button D12 of the help dialogue D1 or without touching the operating portion menu button P22 by simply carrying out the reading operation of the barcode reader 52 as shown in S111 of FIG. 9. The operator thus can appropriately proceed the reagent replacing task by the input with respect to the reagent replacement dialogue D2 displayed on the display input unit 51 in addition to by simply following the audio guidance.

According to the embodiment described above, when the barcode information of the existing reagent container in the acceptance standby state is read again (S107 in FIG. 10: YES), the acceptance standby state of the existing reagent container is canceled. Therefore, even if the existing reagent container is set to the acceptance standby state by mistake, the operator can cancel the acceptance standby state of the existing reagent container with a simple and easy operation of again reading the barcode information of the relevant existing reagent container.

According to the embodiment described above, “acceptance standby” and “accepted” are displayed in the reagent replacement dialogue D2, and thus the operator can check the progress of the current reagent replacing task by looking at the display input unit 51 during the series of tasks of reagent replacement.

The embodiment of the present invention has been described above, but the embodiment of the present invention is not limited thereto.

For example, in the embodiment described above, a case in which the measurement target of the measurement unit 4 is blood has been described by way of example, but the measurement target of the measurement unit 4 may be urine. In other words, the present invention can be applied to the sample processing system including the measurement unit for measuring urine using the reagent, and the present invention can also be applied to a clinical sample processing system including a measurement unit for measuring other clinical samples using the reagent. The present invention can also be applied to a sample processing system including a smear creating device for creating a smear of the sample using the reagent. The present invention can also be applied to a reagent preparation apparatus for preparing a diluted reagent by mixing high concentration reagent and water. When the present invention is applied to the reagent preparation apparatus, “reagent container” mentioned in the Claims corresponds to the reagent container containing the concentrated reagent before dilution.

Even when the measurement target is another clinical sample, the measurement unit for measuring another clinical sample is connected to the information processing unit 5, similar to the embodiment, and the reagent is replaced using the barcode reader 52, similar to the embodiment. When the smear is created by the smear creating device as well, the smear creating device is connected to the information processing unit 5, similar to the embodiment, and the reagent is replaced using the barcode reader 52, similar to the embodiment.

Furthermore, the sample processing system 1 according to the embodiment includes the inputting unit 21, the pre-processing unit 22, the collecting unit 23, two transportation units 3, and the transportation controller 7 for controlling transportation other than the blood cell analyzer 6. However, not limited thereto, the sample processing system 1 may include only the blood cell analyzer 6 and a transportation unit for allocating the sample rack to the front of the two measurement units 4.

In the embodiment described above, the display input unit 51 of the information processing unit 5 is configured by a touch panel type display, but instead, may be configured by an input unit including a mouse and a keyboard, and a display unit including a display. The audio guidance is output from the speaker 51a integrally installed with the display input unit 51, but is not limited thereto, and may be output from a speaker separately arranged. The speaker 51a may be arranged in the barcode reader 52.

In the embodiment described above, the barcode reader 52 is connected to the main body 50 with a sufficiently long cable, but instead, may be communicably connected to the main body 50 wirelessly.

In the embodiment described above, when the existing reagent container is appropriately input, the audio guidance 1 to 3 are output from the speaker 51a in S113, S115, S116 of FIG. 9, but instead, a mere sound may be output from the speaker 51a. If the new reagent container is appropriately input, the audio guidance 6 is output from the speaker 51a in S126 of FIG. 10, but instead, a mere sound may be output from the speaker 51a. The operator thus can recognize that the barcode information of the existing reagent container or the new reagent container is appropriately input. If a mere sound is output from the speaker 51a, the audio guidance is desirably output from the speaker 51a, as in the embodiment described above, following the sound.

In the embodiment described above, if the new reagent container is usable, the audio guidance 6 including the type and expiration date of the reagent contained in the new reagent container is output from the speaker 51a based on the barcode information of the new reagent container in S126 of FIG. 10. However, not limited thereto, the audio guidance 6 including a lot number and a serial number of the new reagent container may be output.

In the embodiment described above, the audio guidance output in S113 of FIG. 10 includes the remaining number of usage times of the existing reagent container, but in place of or in addition thereto, may include the remaining amount of existing reagent container.

In the embodiment described above, when the reagent is in the acceptance standby state, and barcode information of the new reagent container of the same type as the relevant reagent is read thereafter, such reagent is in the accepted state. The reagent replacing operation is started when the barcode information of the new reagent container is read again. Thus, in the embodiment described above, the reagent replacing operation is started when the barcode information of the new reagent container is read again once after being in the accepted state, but the number of times to read the barcode information with respect to the new reagent container is not limited. For example, if the barcode information of the new reagent container is read again, the audio guidance notifying the start of the reagent replacing operation is output with the registration of the barcode information and the reading of the barcode information of one more time, and the reagent replacing operation may be started by the reading of the barcode information one more time thereafter.

In the embodiment described above, the reagent replacing operation is started when the barcode information of the new reagent container is read again once after being in the accepted state for each reagent, but the reagent replacing operation may be started for all reagents in the accepted state when the barcode information of the new reagent container is read again once for all the reagents in the accepted state. Furthermore, the reagent replacing operation may be started for all reagents in the accepted state when the barcode information of the new reagent container is read again once for any reagent in the accepted state.

In the embodiment described above, when the new reagent container is in the accepted state, the audio guidance including the phrase “this reagent will be registered in the reagent management DB by reading the barcode of the reagent again” may be output from the speaker 51a. In this case, when the barcode information of the new reagent container is read again, the registration of the new reagent container may be carried out, and then the audio guidance 6 may be output from the speaker 51a. The reagent replacing operation is started when the barcode information of the new reagent container is read again.

In the above embodiment, the measurement is interrupted when the reagent is in the accepted state, as shown in S202 and S203 of FIG. 13A, but instead, the measurement may be interrupted when the reagent is in the acceptance standby state. In the above embodiment, the measurement of the sample scheduled to be measured next is interrupted when the existing reagent container is in the accepted state, but the timing to interrupt the measurement is not limited thereto. For example, the measurement of the sample scheduled to be measured two times after may be interrupted when the existing reagent container is in the accepted state.

In the above embodiment, the reagent replacing operation of the new reagent container is started when the barcode information of the new reagent container in the accepted state is read again, but instead, for example, the reagent replacing operation of all new reagent containers in the accepted state may be started when the ID card for identifying the operator is read by the barcode reader 52.

In the above embodiment, the barcode labels 31a to 36a are attached to the reagent containers 31 to 36, but are not limited thereto, and a two-dimensional barcode and RFID (Radio Frequency IDentification) tag may be attached. In this case, the information regarding the reagent is stored in the two-dimensional barcode, the RFID tag, and the like, similar to the barcode information described above. The main body 50 of the information processing unit 5 may be connected with a reader for reading the information regarding the reagent from the two-dimensional barcode or an antenna for reading out the information regarding the reagent from the RFID tag in place of the barcode reader 52.

In the above embodiment, the barcode labels 31a to 36a are attached to the reagent containers 31 to 36 that directly contain the reagent, but is not limited thereto, and merely needs to be attached to a portion that can specify the reagent containers 31 to 36. For example, the barcode labels 31a to 36a may be attached to containers and boxes for accommodating the reagent containers 31 to 36 from the outer side, that is, containers and boxes on the outer side when the reagent container has a multiple structure. The barcode labels 31a to 36a may be attached to a tag (label) attached to the reagent containers 31 to 36. If the reagent containers 31 to 36 are packed in a packing material at the time of conveyance, the barcode labels 31a to 36a may be attached to the packing material or the barcode may be directly printed thereon.

Furthermore, the embodiment of the present invention can be subjected to various modifications appropriately within a scope of a technical concept defined in the Claims.

What is claimed is:

1. A sample processing apparatus, comprising:
a sample processing unit that carries out processing of a sample using a reagent;
an acquiring device that acquires reagent information regarding a reagent in a reagent container provided to the reagent container;
an audio output unit that outputs audio; and
a control device that manages the reagent information of the reagent in a state of being used by the sample processing unit;
wherein the audio output unit outputs audio guidance regarding the acquired reagent information when the acquiring device acquires new reagent information regarding a reagent not in a state of being used by the sample processing unit.

2. The sample processing apparatus according to claim 1, wherein the audio guidance includes audio indicating that acquisition of the new reagent information by the acquiring device is successful.

3. The sample processing apparatus according to claim 2, wherein the audio includes type of reagent corresponding to the new reagent information.

4. The sample processing apparatus according to claim 1, wherein the reagent information includes at least an expiration date of the reagent; and the control device causes the audio output unit to output a warning regarding the expiration date as the audio guidance if the expiration date included in the new reagent information acquired by the acquiring device does not satisfy a predetermined condition.

5. The sample processing apparatus according to claim 1, wherein the control device registers the acquired reagent information as target of management when a predetermined operation using the acquiring device is carried out after the reagent information is acquired by the acquiring device.

6. The sample processing apparatus according to claim 5, wherein the audio guidance includes audio indicating that the predetermined operation is required to register the acquired reagent information in the control device.

7. The sample processing apparatus according to claim 5, wherein the predetermined operation is an operation of again acquiring the reagent information using the acquiring device from the new reagent container, in which the reagent information is acquired.

8. The sample processing apparatus according to claim 1, wherein the control device causes the sample processing unit to execute a reagent replacing operation with respect to the new reagent container, in which the reagent information is acquired, when the predetermined operation using the acquiring device is carried out after the acquired reagent information is registered in the control device.

9. The sample processing apparatus according to claim 8, wherein when the acquired reagent information is registered, the audio output unit outputs audio indicating that the predetermined operation for executing the reagent replacing operation is required.

10. The sample processing apparatus according to claim 1, wherein the control device has a database that stores information of the reagent in a state of being used by the sample processing unit, and registers the new reagent information in the database when the predetermined operation for registering the reagent information is carried out using the acquiring device.

11. The sample processing apparatus according to claim 1, wherein the control device causes the sample processing unit to interrupt a process on a sample after a predetermined processing order scheduled after the acquisition when the new reagent information is acquired by the acquiring device.

12. The sample processing apparatus according to claim 1, wherein if after the audio guidance is output for a first reagent of a predetermined type, the acquiring device again acquires new reagent information regarding a reagent not in a state of being used by the sample processing unit for a second reagent of the same type, the audio output unit again outputs the audio guidance for the second reagent.

13. The sample processing apparatus according to claim 12, wherein the audio guidance includes audio indicating that a new reagent to be replaced is changed.

14. The sample processing apparatus according to claim 1, wherein when the acquiring device acquires existing reagent information regarding a reagent in a state of being used by the sample processing unit, the control device specifies a reagent corresponding to the existing reagent information as target of replacement, and the audio output unit outputs the audio guidance when the acquiring device acquires reagent information of a new reagent of the same type as the reagent set as the target of replacement.

15. The sample processing apparatus according to claim 14, wherein when the acquiring device acquires the existing reagent information, the audio output unit outputs second audio guidance indicating a procedure for proceeding the replacement from an existing reagent corresponding to the existing reagent information to a new reagent using the acquiring device.

16. The sample processing apparatus according to claim 15, wherein the second audio guidance includes audio urging to acquire the reagent information from the new reagent container by means of the acquiring device.

17. The sample processing apparatus according to claim 15, wherein the second audio guidance includes audio indicating that the existing reagent information is appropriately input through the acquiring device.

18. The sample processing apparatus according to claim 15, wherein the second audio guidance includes audio indicating type of the existing reagent set as the target of replacement.

19. The sample processing apparatus according to claim 15, wherein the second audio guidance includes audio notifying remaining amount of the existing reagent.

20. The sample processing apparatus according to claim 19, wherein the remaining amount of the existing reagent is notified by the number of times the existing reagent can be used in the process.

21. The sample processing apparatus according to claim 15, wherein the second audio guidance includes audio regarding an expiration date of the existing reagent.

22. The sample processing apparatus according to claim 14, wherein the control device includes a display unit for displaying a screen; and when the existing reagent information is acquired by the acquiring device, the display unit displays an operation screen for replacing the reagent corresponding to the existing reagent information.

23. The sample processing apparatus according to claim 14, wherein the control device cancels the reagent from the target of replacement when the reagent information of the existing reagent set to be the target of replacement is acquired again by the acquiring device.

24. The sample processing apparatus according to claim 1, wherein the reagent in a state of being used is connected to the sample processing unit, and the reagent not in a state of being used is not connected to the sample processing unit.

25. The sample processing apparatus according to claim 1, wherein the acquiring device is a handy type barcode reader.

26. A reagent information informing method, comprising:
(a) processing a sample using a reagent;
(b) acquiring reagent information regarding a reagent in a reagent container provided to the reagent container; and
(c) outputting audio;
wherein when new reagent information regarding a reagent not in a state of being used in the sample processing step is acquired by an acquiring device in the acquiring step, audio guidance regarding the acquired reagent information is output in the step (c).

27. A sample processing apparatus, comprising:
a sample processing unit that carries out processing of a sample using a reagent;
an acquiring device that acquires reagent information regarding a reagent in a reagent container provided to the reagent container;
an audio output unit that outputs audio; and
a control device that manages the reagent information of the reagent in a state of being used by the sample processing unit;
wherein the audio output unit outputs audio guidance regarding the acquired reagent information when the acquiring device acquires new reagent information regarding a reagent in a state of being used by the sample processing unit.
